(19) United States
(12) Patent Application Publication
(10) Pub. No.: US 2022/0174947 A1
(43) Pub. Date: Jun. 9, 2022

(54) ANTIMICROBIAL MIXTURE CONTAINING 4-(3-ETHOXY-4-HYDROXYPHENYL)BUTAN-2-ONE AND AN ALCOHOL COMPOUND, AND COSMETIC COMPOSITION CONTAINING SAME

(71) Applicant: L'OREAL, Paris (FR)

(72) Inventors: Florence MENARD-SZCZEBARA, Chevilly La Rue (FR); Sylvie CUPFERMAN, Chevilly La Rue (FR); Julien GALVAN, Chevilly La Rue (FR)

(21) Appl. No.: 17/311,391

(22) PCT Filed: Dec. 17, 2019
(86) PCT No.: PCT/EP2019/085792

§ 371 Date: Jun. 7, 2021

(30) Foreign Application Priority Data
Dec. 20, 2018 (FR) 1873695

(51) Int. Cl. A01N 31/16; A01P 1/00; A61K 8/35; A61Q 17/00; A23L 3/3463; A23L 3/3499; C02F 1/50

(52) U.S. Cl. CPC A01N 31/16; A01P 1/00; A61K 8/35; A61Q 17/005; A23V 2002/00; A23L 3/3499; C02F 1/50; C02F 2303/04; A23L 3/34635

(57) ABSTRACT

The invention relates to an antimicrobial mixture containing 4-(3-ethoxy-4-hydroxyphenyl)butan-2-one and an alcohol compound chosen from hydroxyacetophenone, 5-chloro-2-(2,4-dichlorophenoxy)phenol, sorbitan caprylate, the combination of 80% to 90% by weight, relative to the total weight of said combination, of xylityl sesquicaprylate and from 10% to 20% by weight of anhydroxylitol, 4-chloro-3,5-dimethylphenol, and glyceryl caprylate, and also to a cosmetic, pharmaceutical or nutritional composition containing such a mixture.

Application to caring for, making up and cleansing keratin materials; to preserving foods and to water treatment.

The present invention relates to an antimicrobial mixture containing 4-(3-ethoxy-4-hydroxyphenyl)butan-2-one and an alcohol compound, and also to a cosmetic, pharmaceutical or nutritional composition containing such a mixture.

### TECHNICAL FIELD

4-(3-Ethoxy-4-hydroxyphenyl)butan-2-one (ketone compound) is a beneficial substance as a preserving agent for cosmetic compositions, for protecting the compositions against microbial contamination, as described in patent application WO 2011/039445.

However, it is desirable to be able to incorporate said ketone compound in reduced concentration in compositions, notably cosmetic or dermatological compositions, while at the same time maintaining good antimicrobial conservation performance. Combinations of the ketone compound with other compounds that have good antimicrobial efficacy are thus sought for this purpose.

The invention may be better understood on reading the following description accompanied with nonlimiting implementation examples thereof with reference to the appended drawings, in which:

The inventors have discovered, unexpectedly, that the combination of
at least one 4-(3-ethoxy-4-hydroxyphenyl)butan-2-one and salts thereof with an organic or inorganic acid or base, and solvates thereof such as the hydrates; with
at least one alcohol compound chosen from:
   hydroxyacetophenone and salts thereof with an organic or inorganic acid or base, and solvates thereof such as the hydrates;
   5-chloro-2-(2,4-dichlorophenoxy)phenol and salts thereof with an organic or inorganic acid or base, and solvates thereof such as the hydrates;
   sorbitan caprylate and its optical isomers, and solvates thereof such as the hydrates;
   the combination of:
a) 80% to 90% by weight, relative to the total weight of said combination, of xylityl sesquicaprylate and its optical isomers, and solvates thereof such as the hydrates, and
b) from 10% to 20% by weight of anhydroxylitol (especially 1,4-anhydroxylitol) and its optical isomers, and solvates thereof such as the hydrates;
   4-chloro-3,5-dimethylphenol and salts thereof with an organic or inorganic acid or base, and solvates thereof such as the hydrates, and
   glyceryl caprylate and its optical isomers, and solvates thereof such as the hydrates,
      makes it possible to obtain an antimicrobial mixture which has synergistic antimicrobial activity.

More particularly, the combination of
4-(3-ethoxy-4-hydroxyphenyl)butan-2-one; with
an alcohol compound chosen from:
   hydroxyacetophenone preferably 4-hydroxyacetophenone;
   5-chloro-2-(2,4-dichlorophenoxy)phenol;
   sorbitan caprylate;
   the combination of 80% to 90% by weight, relative to the total weight of said combination, of xylityl sesquicaprylate and from 10% to 20% by weight of anhydroxylitol (especially 1,4-anhydroxylitol);
   4-chloro-3,5-dimethylphenol, and
   glyceryl caprylate,
      makes it possible to obtain an antimicrobial mixture which has synergistic antimicrobial activity.

The results of the examples described below show the synergistic antimicrobial activity obtained with the minimum inhibitory concentration (MIC) measurements taken with several mixtures. The antimicrobial activity is considered as being synergistic when the antimicrobial mixture makes it possible to obtain a percentage of strain growth of less than or equal to 25%, or even less than or equal to 20%.

Particularly, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
hydroxyacetophenone and salts thereof with an organic or inorganic base, and solvates thereof such as the hydrates, especially hydroxyacetophenone;
   makes it possible to obtain an antimicrobial mixture with synergistic antimicrobial activity, in particular on molds, notably on Aspergillus niger.

According to one embodiment, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
5-chloro-2-(2,4-dichlorophenoxy)phenol and salts thereof with an organic or inorganic base, and solvates thereof such as the hydrates, especially 5-chloro-2-(2,4-dichlorophenoxy)phenol;
   makes it possible to obtain an antimicrobial mixture with synergistic antimicrobial activity, in particular on the Gram-positive bacterium Staphylococcus aureus.

According to another embodiment, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
sorbitan caprylate and its optical isomers, and solvates thereof such as the hydrates, especially sorbitan caprylate;
   makes it possible to obtain an antimicrobial mixture which has synergistic antimicrobial activity, in particular on molds, notably on Aspergillus niger, and on the Gram-positive bacterium Enterococcus faecalis.

According to another embodiment, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
the combination of 80% to 90% by weight, relative to the total weight of said combination, of a) xylityl sesquicaprylate and its optical isomers, and solvates thereof such as the hydrates, especially xylityl sesquicaprylate, and b) from 10% to 20% by weight of anhydroxylitol (especially 1,4-anhydroxylitol) and its optical isomers, and solvates thereof such as the hydrates, particularly anhydroxylitol (especially 1,4-anhydroxylitol);
   makes it possible to obtain an antimicrobial mixture with synergistic antimicrobial activity, in particular on the Gram-positive bacterium Enterococcus faecalis, on Gram-negative bacteria, notably on Pseudomonas aeruginosa, and on molds, notably on Aspergillus niger.

According to another embodiment, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
4-chloro-3,5-dimethylphenol and salts thereof with an organic or inorganic base, and solvates thereof such as the hydrates, especially 4-chloro-3,5-dimethylphenol;
   makes it possible to obtain an antimicrobial mixture which has synergistic antimicrobial activity, in particular on yeasts, in particular on Candida albicans, and on molds, notably on Aspergillus niger.

According to another embodiment, the combination of:
4-(3-ethoxy-4-hydroxyphenyl)butan-2-one and salts thereof with an organic or inorganic base, and solvates thereof such as the hydrates, especially 4-(3-ethoxy-4-hydroxyphenyl)butan-2-one; with
glyceryl caprylate and its optical isomers, and solvates thereof such as the hydrates, especially glyceryl caprylate;
   makes it possible to obtain an antimicrobial mixture which has synergistic antimicrobial activity, in particular on Gram-positive bacteria, notably on Enterococcus faecalis and Staphylococcus aureus and on Gram-negative bacteria, notably on Pseudomonas aeruginosa.

More precisely, a subject of the invention is an antimicrobial mixture comprising, or constituted of (or consisting of):
4-(3-ethoxy-4-hydroxyphenyl)butan-2-one and salts thereof with an organic or inorganic acid or base, and solvates thereof such as the hydrates; and
an alcohol compound chosen from:
   hydroxyacetophenone (preferably 4-hydroxyacetophenone) and salts thereof with an organic or inorganic base, and solvates thereof such as the hydrates,
   5-chloro-2-(2,4-dichlorophenoxy)phenol and salts thereof with an organic or inorganic base, and solvates thereof such as the hydrates,
   sorbitan caprylate and its optical isomers, and solvates thereof such as the hydrates,
   the combination of 80% to 90% by weight, relative to the total weight of said combination, of xylityl sesquicaprylate and its optical isomers, and solvates thereof such as the hydrates and from 10% to 20% by weight of anhydroxylitol (especially 1,4-anhydroxylitol) and its optical isomers, and solvates thereof such as the hydrates,
   4-chloro-3,5-dimethylphenol and salts thereof with an organic or inorganic base, and solvates thereof such as the hydrates, and
   glyceryl caprylate and its optical isomers, and solvates thereof such as the hydrates.

For the purposes of the present invention, and unless otherwise indicated:

an “organic orinorganic acid salt” is more particularly chosen from salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
an “organic or inorganic base salts” means salts of bases or alkaline agents as defined below, such as alkali metal hydroxides, such as sodium hydroxide, potassium hydroxide, or alkaline earth metal calcium hydroxide, ammonia, amines or alkanolamines.
an “hydroxyacetophenone” means 2-hydroxyacetophenone, 3-hydroxyacetophenone or 4-hydroxyacetophenone, preferably 4-hydroxyacetophenone;
an “alkyl radical” is a linear or branched hydrocarbonyl chain saturated; moreover, the addition salts that may be used in the context of the invention with 4-(3-ethoxy-4-hydroxyphenyl)butan-2-one are especially chosen from addition salts with a cosmetically acceptable base such as basifying agents as defined below, for instance alkali metal hydroxides such as sodium hydroxide, potassium hydroxide, aqueous ammonia, amines or alkanolamines; preferably 4-(3-ethoxy-4-hydroxyphenyl)butan-2-one can be in a salt form such as formula (I′):

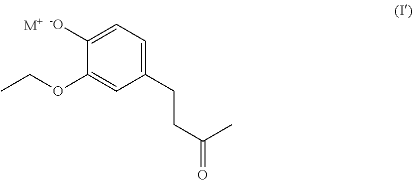

Formula (I′) in which M⁺ represents a cationic counter ion especially an alkali metal such as sodium or potassium, or alkaline earth metal such as calcium or ammonium. the term “at least one” is equivalent to the term “one or more”.

Another subject of the invention is a composition preferably a cosmetic, pharmaceutical or nutritional composition, in a physiologically acceptable medium, comprising:
at least one 4-(3-ethoxy-4-hydroxyphenyl)butan-2-one and salts thereof with an organic or inorganic base, and solvates thereof such as the hydrates; and
at least one alcohol compound chosen from:
   hydroxyacetophenone (preferably 4-hydroxyacetophenone) and salts thereof with an organic or inorganic base, and solvates thereof such as the hydrates
   5-chloro-2-(2,4-dichlorophenoxy)phenol and salts thereof with an organic or inorganic base, and solvates thereof such as the hydrates;
   sorbitan caprylate and its optical isomers, and solvates thereof such as the hydrates;
   the combination of 80% to 90% by weight, relative to the total weight of said combination, of xylityl sesquicaprylate and its optical isomers, and solvates thereof such as the hydrates and from 10% to 20% by weight of anhydroxylitol (especially 1,4-anhydroxylitol) and its optical isomers, and solvates thereof such as the hydrates;
   4-chloro-3,5-dimethylphenol and salts thereof with an organic or inorganic base, and solvates thereof such as the hydrates; and
   glyceryl caprylate and its optical isomers, and solvates thereof such as the hydrates.

A subject of the invention is also a composition comprising said antimicrobial mixture.

The composition may comprise a physiologically acceptable medium. The composition is notably a cosmetic or pharmaceutical or dermatological composition.

The composition may optionally be a nutritional composition (food).

A subject of the invention is also a process for the nontherapeutic cosmetic treatment of keratin materials, comprising the application to the keratin materials of a composition, notably a cosmetic composition, as described previously. The process may be a cosmetic process for caring for or making up or cleansing keratin materials.

A subject of the invention is also a process for conserving a composition, notably comprising a physiologically acceptable medium, in particular a cosmetic or pharmaceutical composition, or a nutritional composition, characterized in that it consists in incorporating into said composition an antimicrobial mixture as described previously.

4-(3-Ethoxy-4-hydroxyphenyl)butan-2-one is a compound of formula:

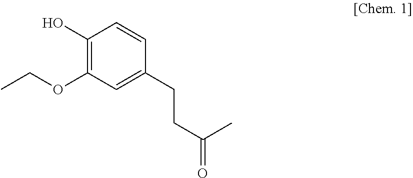

and salts thereof with an organic or inorganic base, and solvates thereof such as the hydrates.

According to a first embodiment, a subject of the invention is an antimicrobial mixture comprising, or constituted of (or consisting of), 4-(3-ethoxy-4-hydroxyphenyl)butan-2-one and hydroxyacetophenone.

Advantageously, 4-(3-ethoxy-4-hydroxyphenyl)butan-2-one and hydroxyacetophenone are present in said mixture in a content such that the 4-(3-ethoxy-4-hydroxyphenyl)butan-2-one/hydroxyacetophenone weight ratio ranges from 0.08 to 0.5, preferably ranges from 0.08 to 0.3, preferentially ranges from 0.08 to 0.25 and more preferentially ranges from 0.15 to 0.25. Such a mixture has good antimicrobial activity on molds, notably on Aspergillus niger.

Preferably, the composition comprising the antimicrobial mixture may comprise 4-(3-ethoxy-4-hydroxyphenyl)butan-2-one in a content ranging from 0.02% to 0.09% by weight, preferentially ranging from 0.03% to 0.75% by weight and better still ranging from 0.04% to 0.06% by weight, relative to the total weight of the composition.

According to a second embodiment, a subject of the invention is an antimicrobial mixture comprising, or constituted of (or consisting of), 4-(3-ethoxy-4-hydroxyphenyl)butan-2-one and 5-chloro-2-(2,4-dichlorophenoxy)phenol.

The antimicrobial mixture may have a 4-(3-ethoxy-4-hydroxyphenyl)butan-2-one/5-chloro-2-(2,4-dichlorophenoxy)phenol weight ratio ranging from 0.3 to 6, preferably ranging from 0.7 to 6, preferentially ranging from 1.5 to 6 and more preferentially ranging from 3 to 6. Such a mixture has good antimicrobial activity on the Gram-positive bacterium Staphylococcus aureus.

Preferably, the composition comprising the antimicrobial mixture may comprise 4-(3-ethoxy-4-hydroxyphenyl)butan-2-one in a content ranging from 0.05% to 0.9% by weight, relative to the total weight of the composition, preferably ranging from 0.08% to 0.9% by weight, preferably ranging from 0.1% to 0.9% by weight, preferably ranging from 0.15% to 0.9% by weight, preferably ranging from 0.2% to 0.9% by weight, preferably ranging from 0.3% to 0.9% by weight, preferably ranging from 0.4% to 0.8% by weight, preferably ranging from 0.4% to 0.7% by weight.

According to a third embodiment, a subject of the invention is an antimicrobial mixture comprising, or constituted of (or consisting of), 4-(3-ethoxy-4-hydroxyphenyl)butan-2-one and sorbitan caprylate.

Sorbitan caprylate is a monoester (CAS No. 95508-00-2); it is notably sold under the name Velsan SC by the company Clariant.

Advantageously, 4-(3-ethoxy-4-hydroxyphenyl)butan-2-one and sorbitan caprylate are present in said mixture in a content such that the 4-(3-ethoxy-4-hydroxyphenyl)butan-2-one/sorbitan caprylate weight ratio ranges from 0.1 to 3.2, preferably from 0.2 to 1.6 and preferably from 0.4 to 1.4.

The antimicrobial mixture may have a 4-(3-ethoxy-4-hydroxyphenyl)butan-2-one/sorbitan caprylate weight ratio ranging from 0.4 to 1.6, preferably from 0.6 to 1.6 and preferentially from 0.8 to 1.4. Such a mixture has good antimicrobial activity on molds, notably on Aspergillus niger. Preferably, the composition comprising the antimicrobial mixture may comprise 4-(3-ethoxy-4-hydroxyphenyl)butan-2-one in a content ranging from 0.02% to 0.09% by weight, preferentially ranging from 0.02% to 0.08% by weight, better still ranging from 0.02% to 0.07% by weight and even better still ranging from 0.02% to 0.06% by weight, relative to the total weight of the composition.

The antimicrobial mixture may have a 4-(3-ethoxy-4-hydroxyphenyl)butan-2-one/sorbitan caprylate weight ratio ranging from 0.1 to 3.2, preferably from 0.2 to 1.6 and preferentially from 0.4 to 1.2. Such a mixture has good antimicrobial activity on the Gram-positive bacterium Enterococcus faecalis. Preferably, the composition comprising the antimicrobial mixture may comprise 4-(3-ethoxy-4-hydroxyphenyl)butan-2-one in a content ranging from 0.05% to 0.04% by weight, preferentially ranging from 0.05% to 0.3% by weight, relative to the total weight of the composition.

According to a fourth embodiment, a subject of the invention is an antimicrobial mixture comprising, or constituted of (or consisting of) 4-(3-ethoxy-4-hydroxyphenyl)butan-2-one and a combination of 80% to 90% by weight, relative to the total weight of said combination, of xylityl sesquicaprylate and from 10% to 20% by weight of anhydroxylitol (also referred to hereinbelow as the xylityl sesquicaprylate/anhydroxylitol combination).

Xylityl sesquicaprylate is a mixture of caprylic acid monoesters and diesters of xylitol; its CAS No. is 181632-90-6. Anhydroxylitol has the CAS No. 53448-53-6.

Use may be made of the combination of xylityl sesquicaprylate and anhydroxylitol (85/15 weight/weight) sold under the name Hebeatol Plus from Chemyunion (INCI name: xylityl sesquicaprylate (and) anhydroxylitol).

The antimicrobial mixture may have a 4-(3-ethoxy-4-hydroxyphenyl)butan-2-one/combination of xylityl sesquicaprylate/anhydroxylitol weight ratio ranging from 0.2 to 13, preferably ranging from 0.2 to 10, preferably ranging from 0.2 to 8, more preferentially ranging from 0.4 to 5, preferably 0.6 to 3. Such a mixture has good antimicrobial activity on Gram-negative bacteria, notably on Pseudomonas aeruginosa. Preferably, the composition comprising the antimicrobial mixture may comprise 4-(3-ethoxy-4-hydroxyphenyl)butan-2-one in a content ranging from 0.05% to 0.9% by weight, preferably from 0.08% to 0.9% by weight, preferably from 0.1% to 0.9% by weight, preferably from 0.2% to 0.9% by weight, preferably from 0.35% to 0.75% by weight, relative to the total weight of the composition.

The antimicrobial mixture may have a 4-(3-ethoxy-4-hydroxyphenyl)butan-2-one/combination of xylityl sesquicaprylate/anhydroxylitol weight ratio ranging from 0.4 to 3.2, preferably from 0.6 to 3, preferably from 1.5 to 2.5. Such a mixture has good antimicrobial activity on the Gram-positive bacterium Enterococcus faecalis. Preferably, the composition comprising the antimicrobial mixture may comprise 4-(3-ethoxy-4-hydroxyphenyl)butan-2-one in a content ranging from 0.05% to 0.4% by weight, preferably from 0.08% to 0.4% by weight, preferably from 0.1% to 0.4% by weight, preferably from 0.15% to 0.4% by weight, preferably from 0.15% to 0.35% by weight, relative to the total weight of the composition.

According to a fifth embodiment, a subject of the invention is an antimicrobial mixture comprising, or constituted of (or consisting of), 4-(3-ethoxy-4-hydroxyphenyl)butan-2-one and 4-chloro-3,5-dimethylphenol.

The antimicrobial mixture may have a 4-(3-ethoxy-4-hydroxyphenyl)butan-2-one/4-chloro-3,5-dimethylphenol weight ratio ranging from 0.1 to 1.4, preferably ranging from 0.15 to 1.4, preferably ranging from 0.15 to 1.2.

The antimicrobial mixture may have a 4-(3-ethoxy-4-hydroxyphenyl)butan-2-one/4-chloro-3,5-dimethylphenol weight ratio ranging from 0.15 to 1.4, preferably from 0.25 to 1.4, preferably from 0.5 to 1.2. Such a mixture has good antimicrobial activity on yeasts, in particular on Candida albicans. Preferably, the composition comprising the antimicrobial mixture may comprise 4-(3-ethoxy-4-hydroxyphenyl)butan-2-one in a content ranging from 0.02% to 0.15% by weight, preferably from 0.04% to 0.15% by weight, preferably from 0.07% to 0.15% by weight, preferably from 0.08% to 0.12% by weight, relative to the total weight of the composition.

The antimicrobial mixture may have a 4-(3-ethoxy-4-hydroxyphenyl)butan-2-one/4-chloro-3,5-dimethylphenol weight ratio ranging from 0.1 to 0.7, preferably from 0.15 to 0.5, preferably from 0.15 to 0.35, preferably from 0.15 to 0.25, preferably from 0.3 to 0.7, preferably from 0.3 to 0.5. Such a mixture has good antimicrobial activity on molds, notably on Aspergillus niger. Preferably, the composition comprising the antimicrobial mixture may comprise 4-(3-ethoxy-4-hydroxyphenyl)butan-2-one in a content ranging from 0.035% to 0.09% by weight, preferably from 0.035% to 0.08% by weight, preferably from 0.04% to 0.07% by weight, preferably from 0.04% to 0.06% by weight, relative to the total weight of the composition.

According to a sixth embodiment, a subject of the invention is an antimicrobial mixture comprising, or constituted of (or consisting of), 4-(3-ethoxy-4-hydroxyphenyl)butan-2-one and glyceryl caprylate.

Glyceryl caprylate is a monoester (CAS No. 26402-26-6).

The antimicrobial mixture may have a 4-(3-ethoxy-4-hydroxyphenyl)butan-2-one/glyceryl caprylate weight ratio ranging from 0.1 to 10. Such a mixture has good antimicrobial activity on the Gram-positive bacterium Staphylococcus aureus. Preferably, the composition comprising the antimicrobial mixture may comprise 4-(3-ethoxy-4-hydroxyphenyl)butan-2-one in a content ranging from 0.05% to 0.9% by weight, relative to the total weight of the composition, preferably ranging from 0.08% to 0.9% by weight, preferably ranging from 0.1% to 0.9% by weight, preferably ranging from 0.15% to 0.9% by weight, preferably ranging from 0.2% to 0.9% by weight, preferably ranging from 0.3% to 0.9% by weight, preferably ranging from 0.4% to 0.8% by weight, preferably ranging from 0.4% to 0.7% by weight.

The antimicrobial mixture may have a 4-(3-ethoxy-4-hydroxyphenyl)butan-2-one/glyceryl caprylate weight ratio ranging from 0.5 to 9, preferably ranging from 0.5 to 7, preferably from 1 to 6 and preferentially from 1 to 3. Such a mixture has good antimicrobial activity on Gram-negative bacteria, notably on Pseudomonas aeruginosa. Preferably, the composition comprising the antimicrobial mixture may comprise 4-(3-ethoxy-4-hydroxyphenyl)butan-2-one in a content ranging from 0.08% to 0.9% by weight, preferably ranging from 0.1% to 0.9% by weight, preferably ranging from 0.2% to 0.9% by weight, preferably ranging from 0.35% to 0.75% by weight, relative to the total weight of the composition.

The antimicrobial mixture may have a 4-(3-ethoxy-4-hydroxyphenyl)butan-2-one/glyceryl caprylate weight ratio ranging from 0.2 to 5, preferably ranging from 0.9 to 4.5. Such a mixture has good antimicrobial activity on the Gram-positive bacterium Enterococcus faecalis. Preferably, the composition comprising the antimicrobial mixture may comprise 4-(3-ethoxy-4-hydroxyphenyl)butan-2-one in a content ranging from 0.05% to 0.4% by weight, preferably from 0.08% to 0.4% by weight, preferably from 0.1% to 0.4% by weight, preferably from 0.15% to 0.4% by weight, preferably from 0.15% to 0.35% by weight, relative to the total weight of the composition.

A subject of the invention is also a composition comprising, in a physiologically acceptable medium, the antimicrobial mixture described previously.

The term “physiologically acceptable medium” means a medium that is compatible with human keratin materials such as the skin, the scalp, the hair and the nails. Said medium may comprise one or more additional ingredients other than the ketone compound and the alcohol compound.

The compound 4-(3-ethoxy-4-hydroxyphenyl)butan-2-one may be present in the composition according to the invention in a content ranging from 0.01% to 5% by weight, relative to the total weight of the composition, preferably ranging from 0.01% to 3% by weight, preferentially ranging from 0.01% to 2.5% by weight, more preferentially ranging from 0.01% to 2% by weight and better still ranging from 0.02% to 0.15% by weight, or else in the contents described previously.

The composition may comprise at least one additional ingredient chosen from water, oils, polyols containing from 2 to 10 carbon atoms, gelling agents, surfactants, film-forming polymers, dyestuffs, fragrances, fillers, UV-screening agents, plant extracts, cosmetic and dermatological active agents, and salts.

The composition according to the invention may comprise an aqueous phase.

The composition may also comprise a polyol that is water-miscible at room temperature (25° C.), notably chosen from polyols notably containing from 2 to 10 carbon atoms, preferably containing from 2 to 6 carbon atoms, such as glycerol, propylene glycol, 1,3-propanediol, butylene glycol, pentylene glycol, hexylene glycol, dipropylene glycol, diethylene glycol or diglycerol. Advantageously, the composition according to the invention comprises 1,3-propanediol, notably in a content ranging from 0.1% to 20% by weight, preferably ranging from 0.1% to 10% by weight and preferentially ranging from 0.5% to 5% by weight, relative to the total weight of the composition.

The compositions according to the invention may be in the form of oil-in-water (O/W) emulsions, water-in-oil (W/O) emulsions or multiple emulsions (triple: W/O/W or O/W/O), oily solutions, oily gels, aqueous solutions, aqueous gels, or solid compositions. These compositions are prepared according to the usual methods.

The compositions according to the invention may be more or less fluid and may have the appearance of a white or coloured cream, an ointment, a milk, a lotion, a serum, a paste or a foam. They may be optionally applied to the skin in aerosol form. They may also be in solid form, for example in the form of a stick or a compact powder.

The composition according to the invention may notably be in the form of:
a makeup product, notably for making up the skin of the face, the body, or the lips or the eyelashes;
an aftershave gel or lotion; a shaving product;
a deodorant (stick, roll-on or aerosol);
a hair-removing cream;
a body hygiene composition such as a shower gel or a shampoo;
a pharmaceutical composition;
a solid composition such as a soap or a cleansing bar;
an aerosol composition also comprising a pressurized propellant;
a hair-setting lotion, a hair-styling cream or gel, a dyeing composition, a permanent-waving composition, a lotion or a gel for combating hair loss, or a hair conditioner;
   a composition for caring for or cleansing the skin.

A subject of the invention is also a process for preparing a composition, notably a cosmetic or pharmaceutical or nutritional composition, comprising a step of mixing 4-(3-ethoxy-4-hydroxyphenyl)butan-2-one, the alcohol compound and one or more additional ingredients, notably cosmetic or pharmaceutical or nutritional ingredients, such as those described previously.

The antimicrobial mixtures of the fourth, fifth and sixth embodiments described previously may be used for preserving foodstuffs (food).

A subject of the invention is also a nutritional composition comprising an antimicrobial mixture chosen from those of the fourth, fifth and sixth embodiments described previously.

The nutritional composition (food) may comprise at least one foodstuff chosen from meats, fish, crustaceans, vegetables, fruit, cereals, eggs, butter, milk, vinegar, water, vegetable oils, sugars, salt, spices, emulsifiers, alcohols, thickeners and honey.

Preferably the 4-(3-ethoxy-4-hydroxyphenyl)butan-2-one is predissolved in a food-grade solvent before being added to the food. Solvents that may be used include ethanol, propylene glycol, isopropyl alcohol and mixtures thereof, optionally combined with water.

The food according to the invention may be, for example, in the form of bread, cake, sauce, candy, a cooked dish, confectionery, jelly, dessert, nougat, drinks, juice, syrup, wine, beer, ravioli, mousse, compote, mayonnaise, mustard, vinaigrette, crisps, sausage, gnocchi, polenta, pancakes, pates, cheeses, flour, delicatessen meats or soup.

The 4-(3-ethoxy-4-hydroxyphenyl)butan-2-one may be present in the food composition in the concentrations described previously.

The synergistic activity of the antimicrobial mixture according to the invention on Gram-negative bacteria, in particular on bacteria of the species Pseudomonas aeruginosa is also of interest in water treatment. This species is notably among the microorganisms commonly sought when a microbiological analysis of a water sample is performed.

The synergistic activity of the antimicrobial mixture according to the invention on fungi, in particular on the species Candida albicans, is also of interest in water treatment. Specifically, fungi represent one of the sources of contamination of water as mentioned in the article “Fungal Contaminants in drinking water regulation?A tale of ecology, exposure, purification and clinical relevance” Int. J. Environ. Res. Public Health 2017, 14, 636.

The synergistic activity of the antimicrobial mixture according to the invention on Gram-positive bacteria, in particular on bacteria of the species Enterococcus faecalis is also of interest in water treatment. Specifically, bacteria of the genus Enterococcus represent one of the sources of contamination of water as mentioned in the article “Enterococci, from commensals to leading causes of drug resistant infection” Michael S. Gilmore et al., 2014, Massachusetts Eye and Ear Infirmary, Boston.

The antimicrobial mixtures of the third, fourth, fifth and sixth embodiments described previously may be used for water treatment.

The present invention also relates to the use of the antimicrobial mixture chosen from those of the third, fourth, fifth and sixth embodiments described previously in water treatment, in which said water is chosen from domestic or industrial waters, waters from aquatic media, swimming pool/spa waters, and waters from air-conditioning systems.

The term “water treatment” refers to a continuous or discontinuous (batch-type) treatment which consists in adding a substance to a water sample to be treated or to a water stream to be treated for the purpose either of preventing the contamination of the water with a contaminant or of partially or totally decontaminating of said contaminant said water to be treated

Preferably, the water treatment performed in the context of the present invention consists in continuously or discontinuously adding a substance to a sample of water to be treated or to a water stream to be treated in order to partially or totally decontaminate of a contaminant said water to be treated.

The contaminant may be a microorganism, in particular a bacterium and/or a fungus.

Even more preferentially, said water treatment is a treatment of water contaminated with one or more microorganisms, preferably with Gram-positive or Gram-negative bacteria or fungi of the species Enterococcus faecalis, Candida albicans or Pseudomonas aeruginosa.

The term “waters of aquatic media” means the waters of lakes, tributary rivers, pools, mainstem rivers, sea or ocean bathing areas, underground waters such as well waters and groundwaters, and aquarium waters.

For the purposes of the present invention, the “domestic or industrial waters” comprise spent waters before they have been treated in a purification plant, waters undergoing treatment in a purification plant, waters before they have been treated in a drinking water plant, waters undergoing treatment in a drinking water plant, and also waters circulating in potable or non-potable urban networks, for instance waters circulating in pipeworks.

The present invention also relates to a continuous or discontinuous water treatment process comprising at least one step of placing a water sample to be treated or a water stream to be treated, said water to be treated being chosen from domestic or industrial waters, waters from aquatic media, swimming pool/spa waters, and waters from air-conditioning systems, in contact with the antimicrobial mixture according to the invention.

Preferably, said step of placing the water to be treated in contact with the antimicrobial mixture according to the invention may notably be performed by injection in liquid form of said compound, by passage through a filter or a filtering cartridge comprising said compound, or by administration in solid form of said compound notably in the form of granules, lozenges or pellets.

The 4-(3-ethoxy-4-hydroxyphenyl)butan-2-one may be used in a proportion of at least 0.06% by weight, preferably at least 0.1% by weight and better still at least 0.5% by weight relative to the total weight of water to be treated. In a preferred embodiment, the compounds of formula (I), alone or as a mixture, may be used in a proportion of at least 1% by weight relative to the total weight of water to be treated.

The 4-(3-ethoxy-4-hydroxyphenyl)butan-2-one may be used in a concentration ranging from 0.06% to 10% by weight, preferably from 0.06% to 10% by weight and better still from 0.06% to 5% by weight relative to the total weight of water to be treated. In a preferred embodiment, the 4-(3-ethoxy-4-hydroxyphenyl)butan-2-one may be used in a concentration ranging from 0.1% to 1% by weight relative to the total weight of water to be treated.

In a preferred embodiment, the 4-(3-ethoxy-4-hydroxyphenyl)butan-2-one is used in combination with an effective amount of at least one organic solvent which may be chosen from ethanol, 1,2-propylene glycol, 1,3-propanediol, PEG-8 (polyethylene glycol containing 8 ethylene glycol units), propylene carbonate, dipropylene glycol, 1,2-hexylene glycol, PEG-4.

Preferably, the organic solvent is chosen from ethanol, 1,2-propylene glycol, 1,3-propanediol, PEG-8 and propylene carbonate.

The solvent may be used in a content ranging from 0.05% to 10% by weight relative to the total weight of the water to be treated, preferably ranging from 0.1% to 5% by weight and preferentially ranging from 0.1% to 2.5% by weight relative to the total weight of the water to be treated.

The invention is illustrated in greater detail in the example that follows. The amounts of the ingredients are expressed as weight percentages.

### EXAMPLES

### Example 1: Determination of the Synergistic Antimicrobial Activity as MIC

The demonstration of a synergistic antimicrobial activity effect with a mixture of 4-(3-ethoxy-4-hydroxyphenyl)butan-2-one (referred to as substance A) and of an alcohol compound (referred to as substance B) was performed by calculating the synergy index (or FIC index) according to the following formula:

FIC Index=(MIC of A with B/MIC of A)+(MIC of B with A/MIC of B [Math. 1]

with:
MIC of A with B: minimum concentration of product A in the combination A+B which makes it possible to obtain an inhibitory effect;
MIC of B with A: minimum concentration of product B in the combination A+B which makes it possible to obtain an inhibitory effect;
MIC of A: minimum inhibitory concentration of product A alone;
MIC of B: minimum inhibitory concentration of product B alone.
   This formula was described for the first time in the article by F. C. Kull, P. C. Eisman, H. D. Sylwestrowka, and R. L. Mayer, Applied Microbiology 9:538-541, 1961.

For each compound tested alone, the MIC is considered as the first concentration which makes it possible to obtain a microbial growth percentage of less than or equal to 25%.

As regards the combinations tested, MIC of A with B and MIC of B with A are the respective concentrations of A and of B in the combinations which make it possible to obtain a microbial growth percentage of less than or equal to 25%.

Interpretation of the FIC Index:

When the FIC index value is less than or equal to 1, it is considered that the combination of test compounds has a synergistic effect.

The summary of the results obtained is presented in the following tables.

The combination of compounds A and B was tested on the following strains or a part thereof: Aspergillus niger, Staphylococcus aureus, Pseudomonas aeruginosa, Enterococcus faecalis, Candida albicans.

The microbial strain Aspergillus niger ATCC 6275, and a double-concentration Sabouraud broth liquid culture medium supplemented with polyoxyethylenated (20 OE) sorbitan monopalmitate (Tween 40 from Croda) and Phytagel© BioReagent were used (i.e. a mixture of 5 g of Phytagel+0.6 g of Tween 40+60 g of Sabouraud broth).

The microbial strain Staphylococcus aureus ATCC 6538 and a double-concentration nutrient broth liquid culture medium were used.

The microbial strain Pseudomonas aeruginosa ATCC 9027 and a double-concentration nutrient broth liquid culture medium were used.

The microbial strain Enterococcus faecalis ATCC 33186 and a double-concentration BHI (Brain Heart Infusion) broth liquid culture medium were used.

The microbial strain Candida albicans ATCC 10231, and a double-concentration Sabouraud broth liquid culture medium were used (i.e. a mixture of 5 g of Phytagel+0.6 g Tween 40+60 g of Sabouraud broth).

A 96-well microplate at an incubation temperature of 32.5° C. is used.

The incubation time of the microplate is:
from 24 to 30 h under aerobic conditions for microbial Aspergillus niger ATCC 6275;
from 18 to 24 h under aerobic conditions for Candida albicans ATCC 10231, Pseudomonas aeruginosa ATCC 9027 and Staphylococcus aureus ATCC 6538;
from 24 to 48 h under aerobic conditions for Enterococcus faecalis ATCC 33186.

Tests

For each compound:
A=4-(3-ethoxy-4-hydroxyphenyl) butan-2-one compound
B=alcohol compound
A 10% (weight/volume) stock solution was prepared by mixing 1 g of compound in 9 ml of aqueous 1% agar solution. Successive dilutions were made with the 1% agar solution.

Tests of Compounds A and B Alone

50 μl of each of the daughter solutions obtained containing compound A or B are added to the microplate wells. 100 μl of Sabouraud liquid nutrient broth inoculated at double concentration with the Aspergillus niger strain and 50 μl of aqueous 1% agar solution are also added thereto.

Tests of Compounds A and B as a Mixture

50 μl of each of the daughter solutions obtained containing compound A and 50 μl of each of the daughter solutions obtained containing compound B are added to the microplate wells. 100 μl of Sabouraud liquid nutrient broth inoculated at double concentration with the strain Aspergillus niger are also added thereto.

Microbial Growth Control

A positive microbial growth control was also prepared. The positive microbial growth control corresponds to a mixture of 100 μl of aqueous 1% agar solution with 100 μl of Sabouraud liquid nutrient broth inoculated at double concentration with the strain Aspergillus niger in the absence of compounds A and B.

Absorbance Control for Compounds A and B Alone

An absorbance control was performed in parallel on compounds A and B alone. This control corresponds to 100 μl of double concentration sterile Sabouraud liquid nutrient broth+100 μl of double concentration compound A or B.

In the three cases (absorbance control, growth control and test), the final volume present in each of the microplate wells is 200 μl.

In the two cases (test and control), the inoculum represents the concentration of the Aspergillus niger strain present in the final volume of the wells (200 μl) and is between 2 and 6×10⁵ cfu/ml of Aspergillus niger.

The minimum inhibitory concentration (MIC) of each compound A and B alone and in combination was determined in a known manner by means of optical density measurements at a wavelength of 620 nm.

The test as described above (tests, absorbance control and growth control) was performed again to test the combination A+B on the following strains Enterococcus faecalis, Staphylococcus aureus, Pseudomonas aeruginosa, Candida albicans, where appropriate.

The following results were obtained with compound B1=4-hydroxyacetophenone:

Aspergillus niger

TABLES 1

| Concentrations tested | | | | |
|---|---|---|---|---|
| (in weight %) | 0 A | 0.025 A | 0.05 A | 0.1 A |
| 0 B1 | | 82 | 43 | 5 |
| 0.0625 B1 | 86 | 71 | 46 | 3 |
| 0.125 B1 | 72 | 73 | 20 | 2 |
| | | | (FIC 0.75) | |
| 0.25 B1 | 79 | 22 | 12 | 5 |
| | | (FIC 0.75) | (FIC 1) | |
| 0.5 B1 | 6 | 2 | −19 | 6 |
| | | (FIC 0.75) | (FIC 1) | |

TABLES 2

| | | MIC of each compound as a | | | |
|---|---|---|---|---|---|
| % MIC of | % MIC of | mixture | | FIC | Ratio |
| A alone | B1 alone | A% | B1 % | Index | A/B1 |
| 0.1 | 0.5 | 0.05 | 0.25 | 1 | 0.2 |

The results obtained show synergistic inhibitory activity for the mixtures:

0.05% of A and 0.25% of B1, i.e. A/B1 ratio=0.2
0.05% of A and 0.125% of B1, i.e. A/B1 ratio=0.4
0.025% of A and 0.25% of B1, i.e. A/B1 ratio=0.1

### Example 2: Determination of the Synergistic Antimicrobial Activity as MIC on Staphylococcus aureus

The demonstration of a synergistic antimicrobial activity effect with a mixture of 4-(3-ethoxy-4-hydroxyphenyl)butan-2-one (referred to as substance A) and of 5-chloro-2-(2,4-dichlorophenoxy)phenol (referred to as substance B2) was performed according to the conditions described in Example 1.

The following results were obtained:

Staphylococcus aureus

TABLES 3

| Concentrations tested | | | | | | |
|---|---|---|---|---|---|---|
| (in weight %) | 0 A | 0.0625 A | 0.125 A | 0.25 A | 0.5 A | 1 A |
| 0 B2 | | 52 | 49 | 52 | 75 | 2 |
| 0.125 B2 | 46 | −8 | −4 | −9 | −12 | −1 |
| | | (FIC | (FIC | (FIC | (FIC | |
| | | 0.56) | 0.63) | 0.75) | 1) | |
| 0.25 B2 | 14 | −7 | 6 | 5 | −10 | −1 |

TABLES 4

| | | MIC of each compound as | | | |
|---|---|---|---|---|---|
| % MIC of A | % MIC of B2 | a mixture | | FIC | Ratio |
| alone | alone | A% | B2% | Index | A/B2 |
| 1 | 0.25 | 0.5 | 0.125 | 1 | 4 |

The results obtained show synergistic inhibitory activity for the mixtures:

i) 0.5% of A and 0.125% of B2, i.e. A/B2 ratio=4
ii) 0.25% of A and 0.125% of B2, i.e. A/B2 ratio=2
iii) 0.125% of A and 0.125% of B2, i.e. A/B2 ratio=1
iv) 0.0635% of A and 0.125% of B2, i.e. A/B2 ratio=0.5

### Example 3

The demonstration of a synergistic antimicrobial activity effect with a mixture of 4-(3-ethoxy-4-hydroxyphenyl)butan-2-one (referred to as substance A) and of sorbitan caprylate (referred to as substance B3) was performed according to the conditions described in Example 1.

Aspergillus niger

TABLES 5

| Concentrations tested | 0 A | 0.025 A | 0.05 A | 0.1 A |
|---|---|---|---|---|
| (in weight %) | | | | |
| 0 B3 | | 45 | 35 | 14 |
| 0.025 B3 | 50 | 42 | 26 | 12 |
| 0.05 B3 | 30 | 19 | 15 | 5 |
| | | (FIC 0.75) | (FIC 1) | |
| 0.1 B3 | −2 | 3 | 8 | 6 |

TABLES 6

| % MIC | % MIC | MIC of each compound | | | |
|---|---|---|---|---|---|
| of A | of B3 | as a mixture | | FIC | Ratio |
| alone | alone | A % | B3 % | Index | A/B3 |
| 0.1 | 0.1 | 0.05 | 0.05 | 1 | 1 |

The results obtained show synergistic inhibitory activity for the mixtures:

i) 0.05% of A and 0.05% of B3, i.e. A/B3 ratio=1
ii) 0.025% of A and 0.05% of B3, i.e. A/B3 ratio=0.5

Enterococcus faecalis

TABLES 7

| Concentrations tested | 0 A | 0.0625 A | 0.125 A | 0.25 A | 0.5 A |
|---|---|---|---|---|---|
| (in weight %) | | | | | |
| 0 B3 | | 70 | 31 | 27 | 14 |
| 0.125 B3 | 134 | 117 | 62 | −16 | −1 |
| 0.25 B3 | 61 | −85 | 4 | 2 | 4 |
| 0.5 B3 | 62 | −8 | 0 | −9 | −12 |
| 1 B3 | 2 | −8 | −5 | −1 | 3 |

TABLES 8

| % MIC | % MIC | MIC of each compound | | | |
|---|---|---|---|---|---|
| of A | of B3 | as a mixture | | FIC | Ratio |
| alone | alone | A % | B3 % | Index | A/B3 |
| 0.5 | 1 | 0.25 | 0.5 | 1 | 0.5 |

The results obtained show synergistic inhibitory activity for the mixtures:

i) 0.25% of A and 0.5% of B3, i.e. A/B3 ratio=0.5

ii) 0.125% of A and 0.5% of B3, i.e. A/B3 ratio=0.25

iii) 0.0625% of A and 0.5% of B3, i.e. A/B3 ratio=0.125

iv) 0.25% of A and 0.25% of B3, i.e. ratio A/B3=1

v) 0.125% of A and 0.25% of B3, i.e. ratio A/B3=0.5

vi) 0.0625% of A and 0.25% of B3, i.e. ratio A/B3=0.25

vii) 0.25% of A and 0.125% of B3, i.e. ratio A/B3=2

### Example 4

The demonstration of a synergistic antimicrobial activity effect with a mixture of 4-(3-ethoxy-4-hydroxyphenyl)butan-2-one (referred to as substance A) and of the combination of xylityl sesquicaprylate and anhydroxylitol (85/15 weight/weight) sold under the name Hebeatol Plus from Chemyunion (referred to as substance B4) was performed according to the conditions described in Example 1.

Pseudomonas aeruginosa

TABLES 9

| Concentrations tested | | | | | | |
|---|---|---|---|---|---|---|
| (in weight %) | 0 A | 0.0625 A | 0.125 A | 0.25 A | 0.5 A | 1 A |
| 0 B4 | | 63 | 60 | 64 | 62 | 7 |
| 0.0625 B4 | 100 | 60 | 27 | 12 | −10 | −8 |
| | | | | (FIC | (FIC | |
| | | | | 0.38) | 0.5) | |
| 0.125 B4 | 57 | 15 | −4 | −9 | 3 | 0 |
| | | (FIC | (FIC | (FIC | (FIC | |
| | | 0.31) | 0.38) | 0.5) | 0.75) | |
| 0.25 B4 | 52 | −5 | −9 | −23 | −22 | −5 |
| | | (FIC | (FIC | (FIC | (FIC 1) | |
| | | 0.56) | 0.63) | 0.75) | | |
| 0.5 B4 | 20 | −5 | −13 | −11 | 7 | −3 |

TABLES 10

| % MIC | % MIC | MIC of each compound | | | |
|---|---|---|---|---|---|
| of A | of B4 | as a mixture | | FIC | Ratio |
| alone | alone | A % | B4 % | Index | A/B4 |
| 1 | 0.5 | 0.5 | 0.25 | 1 | 2 |

The results obtained show synergistic inhibitory activity for the mixtures:

i) 0.5% of A and 0.25% of B4, i.e. A/B4 ratio=2
ii) 0.25% of A and 0.25% of B4, i.e. A/B4 ratio=1
iii) 0.125% of A and 0.25% of B4, i.e. A/B4 ratio=0.5
iv) 0.0625% of A and 0.25% of B4, i.e. A/B4 ratio=0.25
v) 0.5% of A and 0.125% of B4, i.e. ratio A/B4=4
vi) 0.25% of A and 0.125% of B4, i.e. ratio A/B4=2
vii) 0.125% of A and 0.125% of B4, i.e. ratio A/B4=1
viii) 0.0625% of A and 0.125% of B4, i.e. ratio A/B4=0.5
ix) 0.5% of A and 0.0625% of B4, i.e. ratio A/B4=8
x) 0.25% of A and 0.0625% of B4, i.e. ratio A/B4=4

Enterococcus faecalis

TABLES 11

| Concentrations tested | 0 A | 0.0625 A | 0.125 A | 0.25 A | 0.5 A |
|---|---|---|---|---|---|
| (in weight %) | | | | | |
| 0 B4 | | 44 | 39 | 38 | 12 |
| 0.0625 B4 | 45 | 53 | 41 | 30 | 5 |
| 0.125 B4 | 42 | 6 | 7 | 1 | 11 |
| | | (FIC 0.63) | (FIC 0.75) | (FIC 1) | |
| 0.25 B4 | −6 | −10 | 1 | −4 | −4 |

TABLES 12

| % MIC | % MIC | MIC of each compound | | | |
|---|---|---|---|---|---|
| of A | of B4 | as a mixture | | FIC | Ratio |
| alone | alone | A % | B4 % | Index | A/B4 |
| 0.5 | 0.25 | 0.25 | 0.125 | 1 | 0.5 |

The results obtained show synergistic inhibitory activity for the mixtures:

i) 0.25% of A and 0.125% of B3, i.e. A/B4 ratio=2
ii) 0.125% of A and 0.125% of B3, i.e. A/B4 ratio=1
iii) 0.0625% of A and 0.125% of B3, i.e. A/B4 ratio=0.5

### Example 5

The demonstration of a synergistic antimicrobial activity effect with a mixture of 4-(3-ethoxy-4-hydroxyphenyl)butan-2-one (referred to as substance A) and of 4-chloro-3,5-dimethylphenol (referred to as substance B5) was performed according to the conditions described in Example 1.

Candida Albicans

TABLES 13

| Concentrations tested | 0 A | 0.025 A | 0.05 A | 0.1 A | 0.2 A |
|---|---|---|---|---|---|
| (in weight %) | | | | | |
| 0 B5 | | 69 | 54 | 27 | 4 |
| 0.0625 B5 | 73 | 72 | 48 | 22 | 3 |
| 0.125 B5 | 28 | 15 | 11 | 6 | 1 |
| | | (FIC 0.63) | (FIC 0.75) | (FIC 1) | |
| 0.25 B5 | 1 | 0 | 0 | 1 | 0 |

TABLES 14

| % MIC | % MIC | MIC of each compound | | | |
|---|---|---|---|---|---|
| of A | of B5 | as a mixture | | FIC | Ratio |
| alone | alone | A % | B5 % | Index | A/B5 |
| 0.2 | 0.25 | 0.1 | 0.125 | 1 | 0.8 |

The results obtained show synergistic inhibitory activity for the mixtures:

0.1% of A and 0.125% of B5, i.e. A/B5 ratio=0.8
0.05% of A and 0.125% of B, i.e. A/B5 ratio=0.4
0.025% of A and 0.125% of B, i.e. A/B5 ratio=0.2

Aspergillus niger

TABLES 15

| Concentrations tested | 0 A | 0.025 A | 0.05 A | 0.1 A |
|---|---|---|---|---|
| (in weight %) | | | | |
| 0 B5 | | 55 | 32 | 16 |
| 0.0625 B5 | 99 | 31 | 21 | 13 |
| 0.125 B5 | 103 | 40 | 7 | 7 |
| | | | (FIC 0.75) | |
| 0.25 B5 | 31 | 48 | 2 | 7 |
| | | | (FIC 1) | |
| 0.5 B5 | −6 | −27 | −8 | −6 |

TABLES 16

| % MIC | % MIC | MIC of each compound | | | |
|---|---|---|---|---|---|
| of A | of B5 | as a mixture | | FIC | Ratio |
| alone | alone | A % | B5 % | Index | A/B5 |
| 0.1 | 0.5 | 0.05 | 0.25 | 1 | 0.2 |

The results obtained show synergistic inhibitory activity for the mixtures:
i) 0.05% of A and 0.25% of B5, i.e. A/B5 ratio=0.2
ii) 0.05% of A and 0.125% of B5, i.e. A/B5 ratio=0.4

### Example 6

The demonstration of a synergistic antimicrobial activity effect with a mixture of 4-(3-ethoxy-4-hydroxyphenyl)butan-2-one (referred to as substance A) and of glyceryl caprylate (referred to as substance B6) was performed according to the conditions described in Example 1.

Pseudomonas aeruginosa

TABLES 17

| Concentrations | | | | | | |
|---|---|---|---|---|---|---|
| tested (in weight %) | 0 A | 0.0625 A | 0.125 A | 0.25 A | 0.5 A | 1 A |
| 0 B6 | | 80 | 63 | 56 | 61 | −17 |
| 0.1 B6 | 123 | 84 | 58 | 18 | 11 | −7 |
| | | | | (FIC | (FIC | |
| | | | | 0.38) | 0.75) | |
| 0.2 B6 | 109 | 44 | 10 | −5 | −1 | −5 |
| | | | (FIC | (FIC | (FIC 1) | |
| | | | 0.63) | 0.75) | | |
| 0.4 B6 | −40 | 12 | −5 | 2 | 1 | −2 |

TABLES 18

| % MIC | % MIC | MIC of each compound | | | |
|---|---|---|---|---|---|
| of A | of B6 | as a mixture | | FIC | Ratio |
| alone | alone | A % | B6 % | Index | A/B6 |
| 1 | 0.4 | 0.5 | 0.2 | 1 | 2.5 |

The results obtained show synergistic inhibitory activity for the mixtures:

i) 0.5% of A and 0.2% of B6, i.e. A/B6 ratio=2.5
ii) 0.25% of A and 0.2% of B6, i.e. A/B6 ratio=1.25
iii) 0.125% of A and 0.2% of B6, i.e. A/B6 ratio=0.625
iv) 0.5% of A and 0.1% of B6, i.e. A/B6 ratio=5
v) 0.25% of A and 0.1% of B6, i.e. ratio A/B6=2.5

Staphylococcus aureus

TABLES 19

| Concentrations | | | | | | |
|---|---|---|---|---|---|---|
| tested (in weight %) | 0 A | 0.0625 A | 0.125 A | 0.25 A | 0.5 A | 1 A |
| 0 B6 | | 65 | 75 | 79 | 24 | 5 |
| 0.0625 B6 | 83 | 80 | 27 | −1 | 1 | −1 |
| | | | | (FIC | (FIC | |
| | | | | 0.31) | 0.56) | |
| 0.125 B6 | 87 | 58 | 0 | 1 | −9 | 0 |
| | | | (FIC | (FIC | (FIC | |
| | | | 0.25) | 0.38) | 0.63) | |
| 0.25 B6 | 53 | −4 | 1 | 7 | 1 | 0 |
| | | (FIC | (FIC | (FIC | (FIC | |
| | | 0.31) | 0.38) | 0.5) | 0.75) | |
| 0.5 B6 | 48 | 0 | −6 | 4 | 0 | −1 |
| | | (FIC | (FIC | (FIC | (FIC 1) | |
| | | 0.56) | 0.63) | 0.75) | | |
| 1 B6 | 9 | −1 | 0 | 6 | −3 | −6 |

TABLES 20

| % MIC | % MIC | MIC of each compound as a | | | |
|---|---|---|---|---|---|
| of A | of B6 | mixture | | FIC | Ratio |
| alone | alone | A % | B6 % | Index | A/B6 |
| 1 | 1 | 0.5 | 0.5 | 1 | 1 |

The results obtained show synergistic inhibitory activity for the mixtures:

i) 0.5% of A and 0.5% of B6, i.e. A/B6 ratio=1
ii) 0.25% of A and 0.5% of B6, i.e. A/B6 ratio=0.5
iii) 0.125% of A and 0.5% of B6, i.e. A/B6 ratio=0.25
iv) 0.0625% of A and 0.5% of B6, i.e. ratio A/B6=0.125
iv) 0.5% of A and 0.25% of B6, i.e. A/B6 ratio=2
v) 0.25% of A and 0.25% of B6, i.e. ratio A/B6=1
vi) 0.125% of A and 0.25% of B6, i.e. ratio A/B6=0.5
vii) 0.0625% of A and 0.25% of B6, i.e. ratio A/B6=0.25
viii) 0.5% of A and 0.125% of B6, i.e. ratio A/B6=4
ix) 0.25% of A and 0.125% of B6, i.e. ratio A/B6=2
x) 0.125% of A and 0.125% of B6, i.e. ratio A/B6=1
xi) 0.5% of A and 0.0625% of B6, i.e. ratio A/B6=8
xii) 0.25% of A and 0.0625% of B6, i.e. ratio A/B6=4

E. Faecalis

TABLES 21

| Concentrations tested | 0 A | 0.0625 A | 0.125 A | 0.25 A | 0.5 A |
|---|---|---|---|---|---|
| (in weight %) | | | | | |
| 0 B6 | | 35 | 43 | 34 | 0 |
| 0.625 B6 | 38 | 16 | 12 | 4 | −4 |
| | | (FIC | (FIC | (FIC | |
| | | 0.25) | 0.38) | 0.63) | |
| 0.125 B6 | 57 | 5 | −1 | −1 | −7 |
| | | (FIC | (FIC 0.5) | (FIC | |
| | | 0.38) | | 0.75) | |
| 0.25 B6 | 22 | −19 | −12 | −11 | 3 |
| | | (FIC | (FIC | (FIC 1) | |
| | | 0.63) | 0.75) | | |
| 0.5 B6 | 8 | −3 | 1 | −24 | −7 |

TABLES 22

| % MIC | % MIC | MIC of each compound | | | |
|---|---|---|---|---|---|
| of A | of B6 | as a mixture | | FIC | Ratio |
| alone | alone | A % | B6 % | Index | A/B6 |
| 0.5 | 0.5 | 0.25 | 0.25 | 1 | 1 |

The results obtained show synergistic inhibitory activity for the mixtures:

i) 0.25% of A and 0.25% of B6, i.e. A/B6 ratio=1
ii) 0.125% of A and 0.25% of B6, i.e. A/B6 ratio=0.5
iii) 0.0625% of A and 0.25% of B6, i.e. A/B6 ratio=0.25
iv) 0.25% of A and 0.125% of B6, i.e. ratio A/B6=2
v) 0.125% of A and 0.125% of B6, i.e. ratio A/B6=1
vi) 0.0625% of A and 0.125% of B6, i.e. ratio A/B6=0.5
vii) 0.25% of A and 0.0625% of B6, i.e. ratio A/B6=4
viii) 0.125% of A and 0.0625% of B6, i.e. ratio A/B6=2
ix) 0.0625% of A and 0.0625% of B6, i.e. ratio A/B6=1

1. An antimicrobial mixture comprising:
one or more 4-(3-ethoxy-4-hydroxyphenyl)butan-2-one, salt thereof with an organic or inorganic acid or base, solvate thereof and mixture thereof; and
one or more alcohol compounds chosen from:
hydroxyacetophenone and salts thereof with an organic or inorganic base, and solvates thereof;
5-chloro-2-(2,4-dichlorophenoxy)phenol and salts thereof with an organic or inorganic base, and solvates thereof;
sorbitan caprylate, and its optical isomers, and solvates thereof;
the combination of 80% to 90% by weight, relative to the total weight of said combination, of xylityl sesquicaprylate and its optical isomers, and solvates thereof and from 10% to 20% by weight of anhydroxylitol and its optical isomers, and solvates thereof;
4-chloro-3,5-dimethylphenol and salts thereof with an organic or inorganic base, and solvates thereof; and
glyceryl caprylate and its optical isomers, and solvates thereof.

2. The mixture as claimed in claim 1, wherein the alcohol compound is hydroxyacetophenone, salt thereof with an organic or inorganic base, solvate thereof and mixture thereof.

3. The mixture as claimed in claim 1, which has a 4-(3-ethoxy-4-hydroxyphenyl)butan-2-one/hydroxyacetophenone weight ratio ranging from 0.08 to 0.5.

4. The mixture as claimed in claim 1, wherein the alcohol compound is 5-chloro-2-(2,4-dichlorophenoxy)phenol, salt thereof with an organic or inorganic base, salt thereof and mixture thereof.

5. The mixture as claimed in claim 4, which has a 4-(3-ethoxy-4-hydroxyphenyl)butan-2-one/5-chloro-2-(2,4-dichlorophenoxy)phenol weight ratio ranging from 0.3 to 6.

6. The mixture as claimed in claim 1, wherein the alcohol compound is sorbitan caprylate, an optical isomer thereof, solvate thereof and mixture thereof.

7. The mixture as claimed in claim 6, which has a 4-(3-ethoxy-4-hydroxyphenyl)butan-2-one/sorbitan caprylate weight ratio ranging from 0.1 to 3.2.

8. The mixture as claimed in claim 6, which has a 4-(3-ethoxy-4-hydroxyphenyl)butan-2-one/sorbitan caprylate weight ratio ranging from 0.4 to 1.6.

9. The mixture as claimed in claim 6, which has a 4-(3-ethoxy-4-hydroxyphenyl)butan-2-one/sorbitan caprylate weight ratio ranging from 0.1 to 3.2.

10. The mixture as claimed in claim 1, wherein the alcohol compound is a combination of from 80% to 90% by weight, relative to the total weight of said combination, of xylityl sesquicaprylate, an optical isomer thereof, solvate thereof and mixture thereof and from 10% to 20% by weight of anhydroxylitol, an optical isomer thereof, solvate thereof and mixture thereof.

11. The mixture as claimed in claim 10, which has a 4-(3-ethoxy-4-hydroxyphenyl)butan-2-one/combination of xylityl sesquicaprylate/anhydroxylitol weight ratio ranging from 0.2 to 13.

12. The mixture as claimed in claim 10, which has a 4-(3-ethoxy-4-hydroxyphenyl)butan-2-one/combination of xylityl sesquicaprylate/anhydroxylitol weight ratio ranging from 0.4 to 3.2.

13. The mixture as claimed in claim 1, wherein the alcohol compound is 4-chloro-3,5-dimethylphenol, salt thereof with an organic or inorganic base, solvate thereof and mixture thereof.

14. The mixture as claimed in claim 13, which has a 4-(3-ethoxy-4-hydroxyphenyl)butan-2-one/4-chloro-3,5-dimethylphenol weight ratio ranging from 0.1 to 1.4.

15. The mixture as claimed in claim 13, which has a 4-(3-ethoxy-4-hydroxyphenyl)butan-2-one/4-chloro-3,5-dimethylphenol weight ratio ranging from 0.15 to 1.4.

16. The mixture as claimed in claim 13, which has a 4-(3-ethoxy-4-hydroxyphenyl)butan-2-one/4-chloro-3,5-dimethylphenol weight ratio ranging from 0.1 to 0.7.

17. The mixture as claimed in claim 1, wherein the alcohol compound is glyceryl caprylate, an optical isomer thereof, solvate thereof and mixture thereof.

18. The mixture as claimed in claim 17, which has a 4-(3-ethoxy-4-hydroxyphenyl)butan-2-one/glyceryl caprylate weight ratio ranging from 0.1 to 10.

19. The mixture as claimed in claim 17, which has a 4-(3-ethoxy-4-hydroxyphenyl)butan-2-one/glyceryl caprylate weight ratio ranging from 0.5 to 9.

20. The mixture as claimed in claim 17, which has a 4-(3-ethoxy-4-hydroxyphenyl)butan-2-one/glyceryl caprylate weight ratio ranging from 0.2 to 5.

21. A composition in a physiologically acceptable medium, and comprising the mixture as claimed in claim 1.

22. A composition comprising, in a physiologically acceptable medium, an antimicrobial mixture as claimed in claim 1.

23. The composition as claimed in claim 21, which comprises at least one additional ingredient chosen from water, oils, polyols containing from 2 to 10 carbon atoms, gelling agents, surfactants, film-forming polymers, dyestuffs, fragrances, fillers, UV-screening agents, plant extracts, cosmetic and dermatological active agents, and salts.

24. The composition as claimed in claim 21, wherein the 4-(3-ethoxy-4-hydroxyphenyl)butan-2-one, salt thereof with an organic or mineral base, solvate thereof and mixture thereof is present in a content ranging from 0.01% to 5% by weight relative to the total weight of the composition.

25. A nontherapeutic cosmetic treatment process for caring for and/or making up and/or cleansing keratin materials, comprising the application to said keratin materials of a composition as claimed in claim 21.

26. A process for conserving a composition, comprising a physiologically acceptable medium which comprises incorporating into said composition an antimicrobial mixture as defined in claim 1.

27. A process for preserving a nutritional composition, which comprises incorporating into said composition an antimicrobial mixture as defined in claim 1.

28. A process for treating water which comprises incorporating an antimicrobial mixture as defined in claim 1, into the water, wherein the water is chosen from domestic or industrial waters, waters from aquatic media, swimming pool/spa waters, and waters from air-conditioning systems.

29. A continuous or discontinuous water treatment process comprising at least one step of placing a water sample to be treated or a water stream to be treated, said water being chosen from domestic or industrial waters, waters from aquatic media, swimming pool/spa waters, and waters from air-conditioning systems, in contact with the antimicrobial mixture as defined in claim 1.
